(19) United States
(12) Patent Application Publication
(10) Pub. No.: US 2013/0079660 A1
(43) Pub. Date: Mar. 28, 2013

(54) METHOD UTILIZING THERMOGRAPH OF A TEAR FILM FOR GENERATING A QUANTIFIED INDEX

(71) Applicants: O CHANG, Taipei (TW); Chung-Hwa CHANG, Taipei (TW); Po-Hsuan LIU, Taipei (TW); Kenny Hui-Hua CHIANG, Taipei (TW)

(72) Inventors: O CHANG, Taipei (TW); Chung-Hwa CHANG, Taipei (TW); Po-Hsuan LIU, Taipei (TW); Kenny Hui-Hua CHIANG, Taipei (TW)

(21) Appl. No.: 13/241,953

(22) Filed: Sep. 23, 2011

(51) Int. Cl. A61B 5/01

(52) U.S. Cl. 600/549

(57) ABSTRACT

A method utilizing thermograph of a tear film for generating a quantified index includes steps of: capturing a set of tear film thermograph data, deriving parameters from the set of tear film thermograph data, and executing a synthesis calculation based on the derived parameters. The parameters include a temperature distribution of each tear film thermograph, a temperature fall tendency of an open-eye tear film during time variation, a type of a tear film break-up and an occurring time of the tear film break-up, and an upward drifting speed of a temperature color block in the tear film thermograph. The parameters represent the quantity of the aqueous volume of the tear and the stability of the tear film for establishing a reliability of an examination of a diagnosis of a dry eye disease without intruding into a testee's eye.

### BACKGROUND OF THE INVENTION

1. Field of the Invention

The present invention relates to a method for generating a quantified index of a tear film, and more particularly to a method utilizing thermograph of a tear film for generating a quantified index.

2. Description of Related Art

According to the definition of the Dry Eye Workshop (DEWS) updated at June 2007, “Dry eye is a multifactorial disease of the tears and ocular surface that results in symptoms of discomfort, visual disturbance, and tear film instability with potential damage to the ocular surface. It is accompanied by increased osmolarity of the tear film and inflammation of the ocular surface.” The tear film is a protective structure formed on the surface of the cornea of human eye. From inner to outer, the tear film includes a mucous layer, an aquatic layer and an oiliness layer. The oiliness layer is provided for preventing the water from evaporating. The mucous layer is provided for equally distributing the tear and attaching the tear on the surface of the cornea. If the tear is deficiently secreted or excessively evaporated, the dry eye syndrome will occur. The dry eye syndrome is diagnosed with the following examinations:

Schirmer's test: After giving an eye with anesthesia drops thrice, placing a slender filter paper strip on the lower eyelid and observing a moistened length of the filter paper strip after a few minutes.

Tear break-up time (TBUT): placing drops of fluorescent dye in the eye and observing the first tear break-up time under a slit lamp while the eye is open.

The Schirmer's test mainly examines whether the tear is deficiently secreted. The TBUT test analyses the stability of the tear film according to the tear film break-up time. In other words, tear quantity and stability of the tear film are two important indexes for confirming the dry eye syndrome.

Based on page 121 of the 2007 report of the International Dry Eye Workshop, the recommended method for diagnosing the dry eye syndrome “favors technologies that allow changes in tears at the ocular surface to be detected while causing the least disturbance to tear film dynamics during sampling.” Once the tear film dynamics is disturbed, reflex tearing will occur, and the reproducibility of the test will be decreased. As the foregoing tests intrude eyes with either filter paper strip or fluorescent dye, the process of the testing will cause the patient's reflex tearing. Those are not suitable as the recommended method according to the definition of the International Dry Eye Workshop.

To overcome the shortcomings, the present invention tends to provide a method utilizing thermograph of a tear film for generating a quantified index to mitigate or obviate the aforementioned problems.

### SUMMARY OF THE INVENTION

The main objective of the invention is to provide a method utilizing thermograph of a tear film for generating a quantified index and including steps of: capturing a set of tear film thermograph data, deriving parameters from the set of tear film thermograph data and executing a synthesis calculation based on the derived parameters for generating an aqueous volume index of the tear and a stability index of the tear film. The parameters include a temperature distribution of each tear film thermograph, a temperature fall tendency of an open-eye tear film during time variation, a type of a tear film break-up and an occurring time of the tear film break-up, and an upward drifting speed of a temperature color block in the tear film thermograph.

The parameter of the temperature distribution of each tear film thermograph is based on a quantity and a distribution of the temperature color blocks in the tear film thermograph; if the temperature color blocks are equally distributed, an aqueous volume of the tear is determined as adequate; if a number of the temperature color blocks is few and the temperature color blocks are represented in a high temperature range, the aqueous volume of the tear is determined as deficient.

The parameter of the temperature fall tendency of an open-eye tear film during time variation is based on the following. If the temperature fall tendency of an open-eye tear film during time variation is conspicuous and a slope of the temperature fall tendency is steep, the tear film is determined as break-up. If the temperature fall tendency of an open-eye tear film during time variation is not conspicuous and the slope of the temperature fall tendency is flat, the aqueous volume of the tear is determined as deficient.

The parameters of the type of a tear film break-up and the occurring time of the tear film break-up are based on the following. If the type of the tear film break-up shows the temperature color blocks on the tear film thermograph are distributed in a shape of concentric circles or a shape of ellipses, the tear film is determined as stable. If the type of the tear film break-up shows the temperature color blocks on the tear film thermograph are distributed in neither the shape of concentric circles nor the shape of ellipses, the tear film is determined as unstable. If the occurring time of the tear break-up is short, the tear film is determined as instability.

The parameter of the upward drifting speed of the temperature color block in the tear film thermograph is based on a speed of a lower temperature color and flat-shaped block in the tear film thermograph moving upward for determining the aqueous volume of the tear.

Other objects, advantages and novel features of the invention will become more apparent from the following detailed description when taken in conjunction with the accompanying drawings.

### BRIEF DESCRIPTION OF THE DRAWINGS

FIG. 1 is a flow diagram of a method utilizing thermograph of a tear film for generating a quantified index in accordance with the present invention;

FIG. 2 is a schematic diagram showing an aqueous volume index of the tear and a stability index of the tear film;

FIGS. 3A and 3B are tear film thermographs showing temperature distributions on a same tear film;

FIG. 4 is a schematic diagram showing temperature fall tendency of an open-eye tear film during time variation; and

FIGS. 5A and 5B are tear film thermographs showing the types of tear film break-up and occurring time of the tear film break-up.

### DETAILED DESCRIPTION OF PREFERRED EMBODIMENT

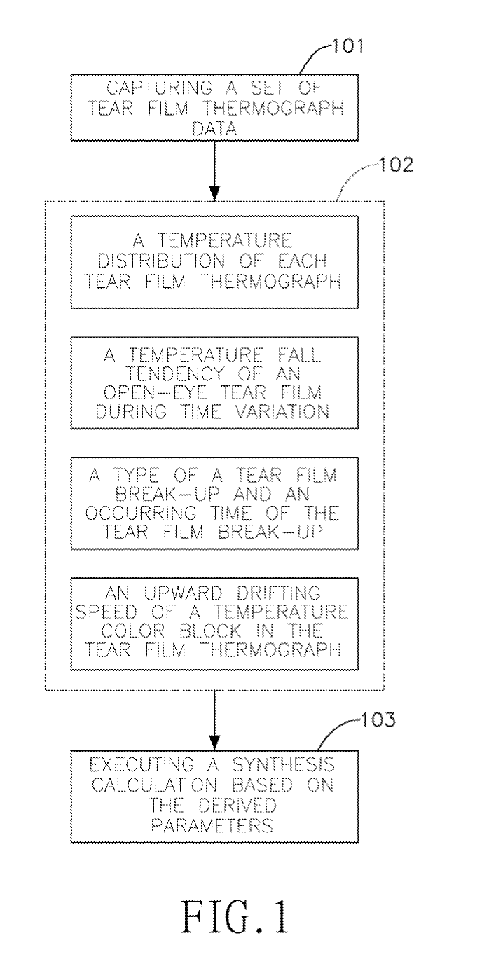

With reference to FIG. 1, a method utilizing thermograph of a tear film for generating a quantified index in accordance with the present invention comprises the steps of: capturing tear film thermographs 101, analyzing the tear film thermographs and deriving parameters from the tear film thermographs 102, and executing a synthesis calculation 103 based on the derived parameters for generating an aqueous volume index of the tear and a stability index of the tear film.

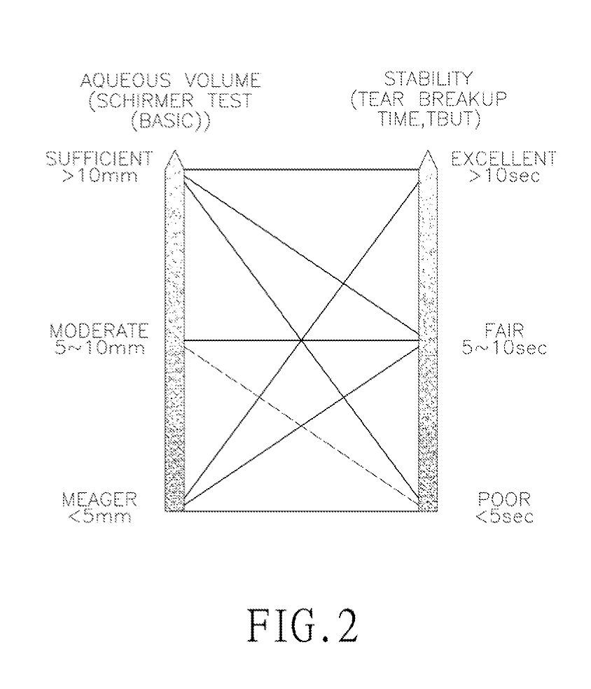

The aqueous volume index of the tear and the stability index of the tear film are shown in FIG. 2. Two arrows are parallel to each other and respectively represent an aqueous volume of the tear and a stability of the tear film. Heights of the arrows respectively represent a quantity of aqueous volume and a quantity of the stability (based on the tear film break-up time).

The step of capturing tear film thermographs 101 utilizes an infrared thermal imaging camera filming a cornea of a testee. Preferably, the tear film thermographs are captured by filming the testee's open eye during a period of six seconds. Each tear film thermograph is sampled in one second. Each tear film thermograph is colorized as different color blocks for showing the temperatures of a captured field due to the infrared thermal imaging camera. Arrangements, shapes, and moving speeds of the color blocks are analyzed and deemed as the parameters.

The parameters, in the step of analyzing the tear film thermographs and deriving parameters from the tear film thermographs 102, include

a temperature distribution of each tear film thermograph;

a temperature fall tendency of an open-eye tear film during time variation;

a type of a tear film break-up and an occurring time of the tear film break-up; and

an upward drifting speed of a temperature color block on the tear film thermograph.

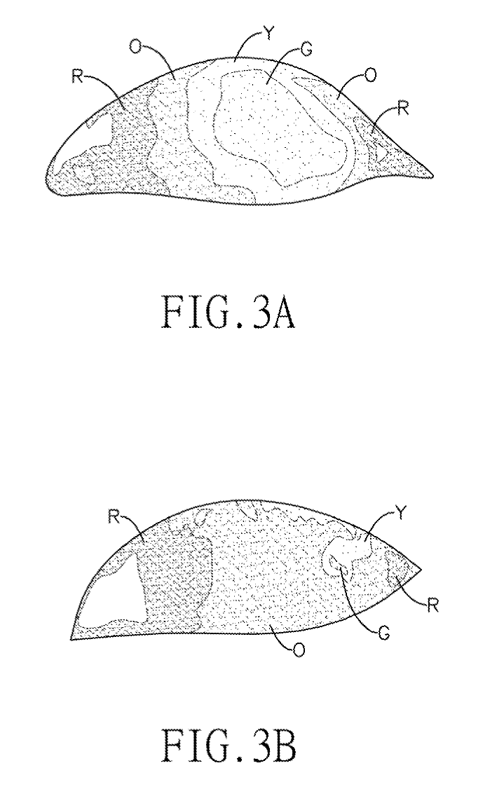

The parameter of the temperature distribution of each tear film thermograph is derived from the following. The step of capturing tear film thermographs 101 provides a series of continuous tear film thermographs of the testee. Each tear film thermograph has different shapes and different colors of the temperature color blocks as shown in FIGS. 3A and 3B. The red color block is represented in the figure as “R”, the orange color block is represented as “O”, the yellow color block is represented as “Y” and the green color block is represented as “G”. The colors of the temperature color blocks show the temperature differences in each tear film thermograph. The temperatures in each tear film thermograph are relatively compared. An average temperature is calculated and is defined as grayscale 125. The average temperature plus 1.8 degree Celsius is defined as a highest temperature and the average temperature minus 1.8 degree Celsius is defined as a lowest temperature, such that the 256 intensities grayscales are represented as 3.6 degree Celsius. The 256 intensities grayscales are divided as eight color blocks. Sequentially, from the lower temperature to the highest temperature, the eight color blocks are black, purple, blue, green, yellow, orange, red and white. The parameter of the temperature distribution of each tear film thermograph is based on a quantity and a distribution of the temperature color blocks in the tear film thermograph. With reference to FIG. 3A, if the temperature color blocks are numerous and are equally distributed, the aqueous volume of the tear is determined as adequate. With reference to FIG. 3B, if a number of the temperature color blocks is few and the temperature color blocks are represented in a high temperature range, the aqueous volume of the tear is determined as deficient.

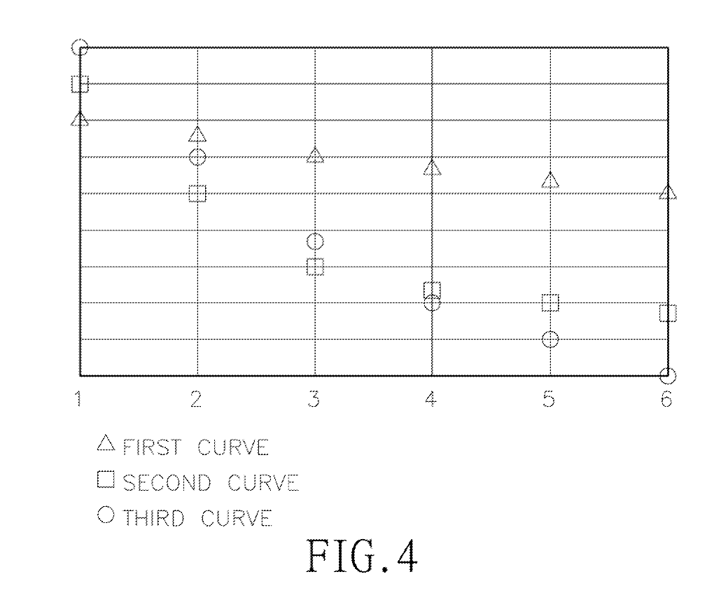

The parameter of the temperature fall tendency of an open-eye tear film during time variation includes a slope and a curvature for showing temperature changes and is derived from the following. With reference to FIG. 4, a temperature fall tendency diagram is based on the six tear film thermographs derived from the step 101. The units in an abscissa axis are time, from the first second to the sixth second. The units in an ordinate axis are temperatures. The temperature fall tendencies of the six tear film thermographs of three different testees during six seconds are represented as a first curve, a second curve, and a third curve. The fall tendency of the first curve is smooth and even and a slope of the temperature fall tendency is flat, representing that the aqueous volume of the tear is adequate and the tear film is stable. The fall tendency of the second curve is not conspicuous and the slope of the temperature fall tendency is not precipitous, representing that the aqueous volume is deficient and the tear film is still stable. The fall tendency of the third curve is conspicuous and precipitous and the slope of the temperature fall tendency is steep, representing that the tear film is broken up. Preferably, if the slope of the fall tendency is higher than 0.141 degree Celsius in each second (10 grayscales in each second), the slope is defined as steep.

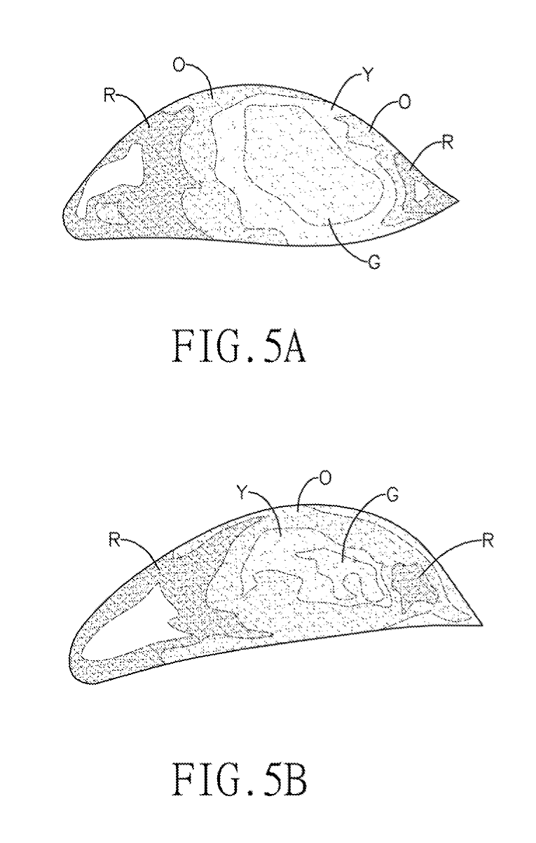

With reference to FIGS. 5A and 5B, the parameters of the type of a tear film break-up and the occurring time of the tear film break-up are based on the following. With reference to FIG. 5A, if the type of the tear film break-up shows the temperature color blocks on each tear film thermograph are distributed in a shape of concentric circles or a shape of ellipses, the tear film is determined as stable. With reference to FIG. 5B, if the type of the tear film break-up shows the temperature color blocks on the tear film thermograph are distributed in neither the shape of concentric circles nor the shape of ellipses, the tear film is determined as unstable. Furthermore, the occurring time of the tear film break-up shows an instability of the tear film, if the occurring time of the tear break-up is short, the tear film is determined as instability. Preferably, the occurring time of the tear break-up is less than 5 seconds, the occurring time of the tear break-up is defined as short.

The parameter of the upward drifting speed of the temperature color block on the tear film thermograph is derived from the following. A driving movement of the temperature color block is caused by a momentum of an upwardly opening eyelid. When the eye is opened, the upper eyelid is moved upward such that the tear film is waved and a wavefront of the tear film is drifted upwardly until to a top of the tear film. In a normal state, the wave front of the tear film takes less than one second to arrive the top of the tear film. The wavefront speed of the tear film is related to the aqueous volume of the tear film. If an upward drifting speed of a lower temperature color and flat-shaped block on the tear film is fast and the lower temperature color and flat-shaped block takes less than one second to reach a top of the tear film, the aqueous volume of the tear is determined as adequate. If the upward drifting speed of the lower temperature color and flat-shaped block on the tear film and the lower temperature color and flat-shaped block takes more than one second to reach the top of the tear film, the aqueous volume of the tear is determined as deficient. Because of each tear film thermograph is sampled in one second, when the wavefront of the tear film is appeared on the tear film thermograph, the upward drifting speed of the lower temperature color and flat-shaped block on the tear film is defined as slow.

When the above parameters are derived, the step of synthesis calculating 103 can be executed. The step of synthesis calculating 103 includes various crossing calculations based on the derived parameters. The techniques of calculations can be a multivariate statistical analysis (like Regression Analysis, Multi-Dimensional Scaling Analysis, etc.) or an artificial neural network.

The parameters represent the quantity of the aqueous volume of the tear and the stability of the tear film. The synthesis calculation based on the derived parameters can generate the aqueous volume index of the tear and the stability index of the tear film for establishing a reliability of an examination of a diagnosis of a dry eye disease. The method in accordance with the present invention utilizes the infrared thermal imaging camera without contacting the cornea of the testee's eye and without intruding into the testee's eye, such that the testee will not feel any discomfort or fear. Furthermore, a disturbance to tear film dynamics can be avoided to enhance the reliability.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A method utilizing thermograph of a tear film for generating a quantified index, comprising steps of:
capturing a set of tear film thermograph data;
deriving parameters from the set of tear film thermograph data, the parameters including
a temperature distribution of each tear film thermograph;
a temperature fall tendency of an open-eye tear film during time variation;
a type of a tear film break-up and an occurring time of the tear film break-up; and
an upward drifting speed of a temperature color block in the tear film thermograph; and
executing a synthesis calculation based on the derived parameters for generating an aqueous volume index of the tear and a stability index of the tear film.

2. The method utilizing thermograph of a tear film for generating a quantified index as claimed in claim 1, wherein the parameter of the temperature distribution of each tear film thermograph is based on a quantity and a distribution of the temperature color blocks in the tear film thermograph;
if the temperature color blocks are equally distributed, an aqueous volume of the tear is determined as adequate; and
if a number of the temperature color blocks is few and the temperature color blocks are represented in a high temperature range, the aqueous volume of the tear is determined as deficient.

3. The method utilizing thermograph of a tear film for generating a quantified index as claimed in claim 1, wherein the parameter of the temperature fall tendency of an open-eye tear film during time variation is based on:
if the temperature fall tendency of an open-eye tear film during time variation is conspicuous and a slope of the temperature fall tendency is steep, the tear film is determined as break-up; and
if the temperature fall tendency of an open-eye tear film during time variation is not conspicuous and the slope of the temperature fall tendency is flat, the aqueous volume of the tear is determined as deficient.

4. The method utilizing thermograph of a tear film for generating a quantified index as claimed in claim 1, wherein the parameters of the type of the tear film break-up and the occurring time of the tear film break-up are based on:
if the type of the tear film break-up shows the temperature color blocks on the tear film thermograph are distributed in a shape of concentric circles or a shape of ellipses the tear film is determined as stable;
if the type of the tear film break-up shows the temperature color blocks on the tear film thermograph are distributed in neither the shape of concentric circles nor the shape of ellipses, the tear film is determined as unstable; and
if the occurring time of the tear break-up is short, the tear film is determined as instability.

5. The method utilizing thermograph of a tear film for generating a quantified index as claimed in claim 1, wherein the parameter of the upward drifting speed of the temperature color block in the tear film thermograph is based on a speed of a lower temperature color and flat-shaped block in the tear film thermograph moving upward for determining the aqueous volume of the tear.

6. The method utilizing thermograph of a tear film for generating a quantified index as claimed in claim 1, wherein the synthesis calculation is utilized by a multivariate statistical analysis.

7. The method utilizing thermograph of a tear film for generating a quantified index as claimed in claim 1, wherein the synthesis calculation is utilized by an artificial neural network.
